(19) United States
(12) Patent Application Publication
(10) Pub. No.: US 2025/0204748 A1
(43) Pub. Date: Jun. 26, 2025

(54) MEDICAL DEVICES AND RELATED METHODS

(71) Applicant: Boston Scientific Scimed, Inc., Maple Grove, MN (US)

(72) Inventor: Michael S.H. CHU, Brookline, MA (US)

(73) Assignee: Boston Scientific Scimed, Inc., Maple Grove, MN (US)

(21) Appl. No.: 18/985,509

(22) Filed: Dec. 18, 2024

Related U.S. Application Data
(60) Provisional application No. 63/612,696, filed on Dec. 20, 2023.

(51) Int. Cl. A61B 1/00; A61B 1/015

(52) U.S. Cl. CPC A61B 1/00068; A61B 1/015

(57) ABSTRACT

Medical devices and related methods are described, including a medical device that may include a handle including a housing, a first lumen extending distal to the housing, and a second lumen extending distal to the housing. The medical device may further include a switch assembly coupled to at least one of the first lumen or second lumen. The switch assembly may include a first inlet, a second inlet, a first outlet, and a second outlet. The first inlet may be configured to receive a source of fluid and the second inlet may be configured to receive a source of suction. The switch assembly may be movable between a first configuration in which the first inlet may be in communication with the first outlet and the first lumen, and a second configuration in which the second inlet may be in communication with the first outlet and the first lumen.

### CROSS-REFERENCE TO RELATED APPLICATIONS

This application claims the benefit of priority to U.S. Provisional Application No. 63/612,696, filed on Dec. 20, 2023, which is incorporated by reference herein in its entirety.

### TECHNICAL FIELD

Various aspects of this disclosure relate generally to medical devices and methods of use thereof. In particular, medical devices and methods useful for endoscopic procedures.

### BACKGROUND

During medical procedures, such as endoscopy and/or ureteroscopy procedures, an operator may position a medical device at a target treatment site in a body lumen of a patient, and extend laser fibers through a working channel of the medical device to break up kidney stones into stone fragments/dust. Use of laser fibers to break up kidney stones in a body lumen may increase the body lumen temperature. Additionally, stone fragments/dust of varying sizes may present challenges to the medical device during the removal of the stone fragments/dust.

### SUMMARY

According to some aspects of the present disclosure, a medical device may include a handle including a housing, a first lumen extending distal to the housing, a second lumen extending distal to the housing, and a switch assembly coupled to at least one of the first lumen or second lumen. The switch assembly may include a first inlet, a second inlet, a first outlet, and a second outlet, the first inlet being configured to receive a source of fluid and the second inlet being configured to receive a source of suction. The switch assembly may be movable between a first configuration in which the first inlet may be in communication with the first outlet and the first lumen and a second configuration in which the second inlet may be in communication with the first outlet and the first lumen.

According to some aspects, the medical device may further include a shaft extending distally from the housing. The shaft may define the first lumen and the second lumen, and the switch assembly may be coupled to the housing of the handle. In some examples, the switch assembly may include a first body that includes the first inlet and the second inlet. The switch assembly may include a second body that includes the first outlet and the second outlet, and the first body may be movable relative to the second body. In some examples, the first body may be rotatable relative to the second body to transition between the first configuration and the second configuration. In some examples, the first body may be rotatable about 60 degrees to about 180 degrees relative to the second body to transition between the first configuration and the second configuration. In some examples, the first body may include a recess for receiving the second body. The recess may include a protrusion configured to engage a groove of the second body. In some examples, the first body may include a first stop and a second stop, and the second body may include a tab configured to engage and move between the first stop and the second stop. In some examples, the switch assembly may include a gasket configured to form a seal between the first body and the second body. In some examples, the second body may include a protrusion configured to be received within an aperture of the first body to secure the second body to the first body. In some examples, the first body may be axially movable relative to the second body to transition the medical device between the first configuration and the second configuration. In some examples, the second body may include the first inlet, the second inlet, the first outlet, and the second outlet. Moving the first body relative to the second body may control fluid communication between the first inlet and one of the first outlet or the second outlet, and may control fluid communication between the second inlet and one of the first outlet or the second outlet. In some examples, the first body may include a plurality of channels. The first inlet may be configured to couple to the first outlet and the second outlet via a first set of channels of the plurality of channels. The second inlet may be configured to couple to the first outlet and the second outlet via a second set of channels of the plurality of channels. In some examples, at least one channel of the first set of channels and at least one of channel of the second set channels may be blocked in the first configuration and in the second configuration. In some examples, each of the first set of channels and the second set of channels may include a channel extending through the first body and a channel formed on an outer surface of the first body.

According to some aspects, the medical device may include a handle including a housing and a shaft extending distally from the handle. The shaft may define a first lumen that terminates at a first opening and a second lumen that terminates at a second opening. The medical device may further include a switch assembly coupled to the housing and the switch assembly may include a first body movably coupled to a second body. The switch assembly may include a first inlet configured to receive a source of a fluid, a second inlet configured to receive a source of suction, a first outlet in communication with the first lumen, and a second outlet in communication with the second lumen. The switch assembly may be movable between a first configuration in which the first inlet may be in communication with the first outlet and the first lumen and a second configuration in which the first inlet may be in communication with the second outlet and the second lumen. In some examples, the first opening may be at a distalmost end of the shaft and the second opening may be proximal to the first opening. In some examples, the second body may include the first inlet, the second inlet, the first outlet, and the second outlet, and the first body may be axially movable within the second body. In some examples, the first body may include a plurality of channels parallel to each other. Moving the first body relative to the second body may establish fluid communication between the first inlet and the first outlet via one of the plurality of channels. Further moving the first body relative to the second body may establish fluid communication between the first inlet and the second outlet via another one of the plurality of channels.

According to some aspects, the medical device may include a handle including a housing and a shaft extending distally from the handle. The shaft may define a first lumen that terminates at a first opening and a second lumen that terminates at a second opening proximal to the first opening. The medical device may further include a switch assembly coupled to the housing and the switch assembly may include a first body movably coupled to a second body. The switch assembly may further include a first inlet configured to receive a source of a fluid, a second inlet configured to receive a source of suction, a first outlet in communication with the first lumen, and a second outlet in communication with the second lumen. The switch assembly may be movable between a first configuration and a second configuration by rotating or axially moving the first body relative to the second body. In the first configuration, the first inlet may be in communication with the first outlet and the first lumen. In the second configuration, the first inlet may be in communication with the second outlet and the second lumen. In some examples, the first body may include the first inlet and the second inlet. The second body may include the first outlet and the second outlet, and the first body may be rotatable relative to the second body.

### BRIEF DESCRIPTION OF FIGURES

The accompanying drawings, which are incorporated in and constitute a part of this application, illustrate exemplary aspects of this disclosure and together with the description, serve to explain the principles of the present disclosure.

FIGS. 1A-1D illustrate an exemplary medical device, in accordance with some aspects of this disclosure.

FIGS. 2A and 2B illustrate features of an exemplary switch assembly, in accordance with some aspects of this disclosure.

FIG. 3 illustrates features of another exemplary switch assembly, in accordance with some aspects of this disclosure.

FIGS. 4A and 4B illustrate features of another exemplary switch assembly, in accordance with some aspects of this disclosure.

FIG. 5 illustrates features of yet another exemplary switch assembly, in accordance with some aspects of this disclosure.

FIG. 6 illustrates features of an exemplary medical device handle, in accordance with some aspects of this disclosure.

FIG. 7 illustrates features of an exemplary switch assembly, in accordance with some aspects of this disclosure.

FIGS. 8A-8C illustrate features of another exemplary switch assembly, in accordance with some aspects of this disclosure.

FIGS. 9A-9C illustrate features of another exemplary switch assembly, in accordance with some aspects of this disclosure.

FIG. 10 illustrates features of an exemplary medical system, in accordance with some aspects of this disclosure.

### DETAILED DESCRIPTION

Particular aspects of the present disclosure are described in greater detail below. The terms and definitions provided herein control, if in conflict with terms and/or definitions incorporated by reference.

The terms “proximal” and “distal” are used herein to refer to the relative positions of the components of exemplary medical devices. As used herein, “proximal” refers to a position relatively closer to the exterior of the body or closer to an operator using the medical device. In contrast, “distal” refers to a position relatively further away from the operator using the medical device, or closer to the interior of the body.

As used herein, the terms “comprises,” “comprising,” “including,” “includes,” “having,” “has,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” Relative terms such as “about,” “substantially,” and “approximately,” etc., are used to indicate a possible variation of +10% of the stated numeric value or range.

Although ureteroscopes are referenced herein for illustration purposes, it will be appreciated that the disclosure encompasses any suitable medical device configured to allow an operator to access and view internal body anatomy of a subject (e.g., patient) and/or to deliver medical instruments, such as, for example, biopsy forceps, graspers, baskets, snares, probes, scissors, retrieval devices, lasers, and other tools, into the subject's body. The medical devices herein may be inserted into a variety of body lumens and/or cavities, such as, for example, the urinary tract or gastrointestinal tract. It will be appreciated that, unless otherwise specified, bronchoscopes, duodenoscopes, endoscopes, gastroscopes, endoscopic ultrasonography (“EUS”) scopes, colonoscopes, ureteroscopes, bronchoscopes, laparoscopes, cystoscopes, aspiration scopes, sheaths, catheters, or any other suitable delivery device or medical device may be used in connection with the features described herein.

Features of the medical devices herein may facilitate removal of stone fragments/dust of varying sizes and/or reduce a temperature of a body lumen during a medical procedure (e.g., laser lithotripsy), among other aspects. According to some aspects of the present disclosure, the medical device may include a handle coupled to a shaft, the shaft defining a first lumen and a second lumen, e.g., to provide for delivery of fluid and/or suction at target site. For example, the first lumen may be used to deliver fluid while the second lumen is used to provide suction, or vice versa. The fluid and suction may be delivered simultaneously. Optionally, the first and second lumens may be parallel to each other along the shaft. The first and second lumens may be coupled to a switch assembly that is, in turn, coupled to (or configured to be coupled to) a source of fluid and a source of suction. The switch assembly may allow an operator of the medical device to select which of the first lumen or second lumen delivers fluid and which of the first or second lumen delivers suction, e.g., by transitioning between a first configuration of the switch assembly and a second configuration of the switch assembly. Such a transition may be performed via the switch assembly, without disrupting or changing coupling between the first and second lumens and the switch assembly, and without disrupting or changing coupling of a source of fluid and a source of suction to the switch assembly.

In some aspects, the first lumen may terminate at a distal opening (e.g., distal-facing opening) of a distal end face of the shaft, and the second lumen may terminate at an opening proximal to the first lumen. In some aspects, the second lumen may terminate at a side-facing opening of the shaft, proximal to the distal opening of the first lumen. Having one lumen terminate proximal to the other lumen may facilitate various functions, such as permitting simultaneous fluid and suction at a target site without interference between the two. Further, for example, having one of the lumens terminal proximal to the distal end of the shaft may provide sufficient room for various components at the distal end of the medical device (e.g., imaging components and devices, light sources, etc.) without enlarging the profile of the shaft of the medical device. In some examples, the first lumen and the second lumen may terminate at the distal end of the shaft and/or a distal end of the first lumen may be aligned with a distal end of the second lumen.

As mentioned above, the medical device may include a switch assembly to allow an operator to switch or exchange functions (e.g., suction, irrigation, etc.) of the two lumens. The switch assembly may be coupled to the handle of the medical device, e.g., coupled to a housing of the handle, and accessible to the operator to transition between the first and second configurations of the switch assembly, e.g., the operator using their hand to move one body of the switch assembly relative to another body of the switch assembly.

For example, the switch assembly may include at least a first inlet and second inlet, and at least a first outlet and a second outlet. One of the first outlet or the second outlet may be coupled to, and in communication with, the first lumen which the other of the first outlet or the second outlet may be coupled to, and in communication with, the second lumen. The first inlet and the second inlet may be coupled to (or configured to couple to) a source of fluid and a source of suction, respectively, or different sources of fluid. Thus, depending on the configuration of the switch assembly, the source of fluid may be in communication with one of the first lumen or the second lumen, which the source of suction may be in communication with the other of the first lumen or the second lumen. Different examples of switch assemblies are discussed below and illustrated in the figures, although the present disclosure contemplates additional variations and combinations of features to those illustrated in the figures, as discussed herein.

For example, in a first configuration of the switch assembly, the first lumen may be used to deliver a fluid (or other material) to a target site (e.g., a target treatment site), while the second lumen may be used to apply suction (or deliver a different fluid or other material) to the target site. In a second configuration of the switch assembly, the supply of fluid (or other material) formerly in communication with the first lumen may instead be in communication with the second lumen, and the supply of suction (or different fluid or material) formerly in communication with the second lumen may instead be in communication with the first lumen. In this way, for example, the operator may control whether, when, and for what purpose each of the first lumen and second lumen is used, without decoupling the source of fluid or suction from the medical device. For various medical procedures, one configuration may be desirable over the other (e.g., proving a fluid at the distal-most end of the shaft while applying suction via an opening proximal to the distal-most end of the shaft), or it may be desirable to switch back and forth between the two configurations, e.g., during a medical procedure or between different steps of a medical procedure.

In some exemplary medical procedures, an operator (e.g., a physician or other medical professional) may deliver a fluid through the first lumen and out of the distal-facing opening to suspend relatively smaller stone fragments/dust (e.g., having a diameter less than approximately 250 μm) at a target site (e.g., a target site within the urinary tract, such as within the kidney), while suction may be applied through the second lumen to pull the fluid and/or stone fragments/dust from the target site into the proximal opening (e.g., proximal side-facing opening) for removal via the second lumen. The operator may use the switch assembly to alternate the use of the first and second lumens, e.g., for removing relatively larger stone fragments/dust (e.g., having a diameter greater than approximately 250 μm and less than 1 mm). For example, the fluid may be delivered through the second lumen and out of the proximal opening (e.g., proximal side-facing opening), while suction may be applied through the first lumen to pull the fluid and/or stone fragments/dust into the distal-facing opening for removal via the first lumen. In this configuration, the operator may aim, maneuver, and/or advance the distal end of the shaft towards the stone fragments/dust and directly suction the stone fragments/dust into the first lumen via the distal-facing opening. Additionally, switching from delivering the fluid through the distal-facing opening via the first lumen to delivering the fluid through the proximal opening via the second lumen may change a velocity and a direction of the fluid, which may reduce scattering effects of the stone fragments/dust.

It will be appreciated that reference to any particular procedure is provided only for convenience and not intended to limit the disclosure. A person of ordinary skill in the art would recognize that the concepts underlying the disclosed devices and application methods may be utilized in any suitable procedure, medical or otherwise. Reference will now be made in detail to examples of the present disclosure described above and illustrated in the accompanying drawings. Wherever possible, the same reference numbers will be used throughout the drawings to refer to the same or like parts.

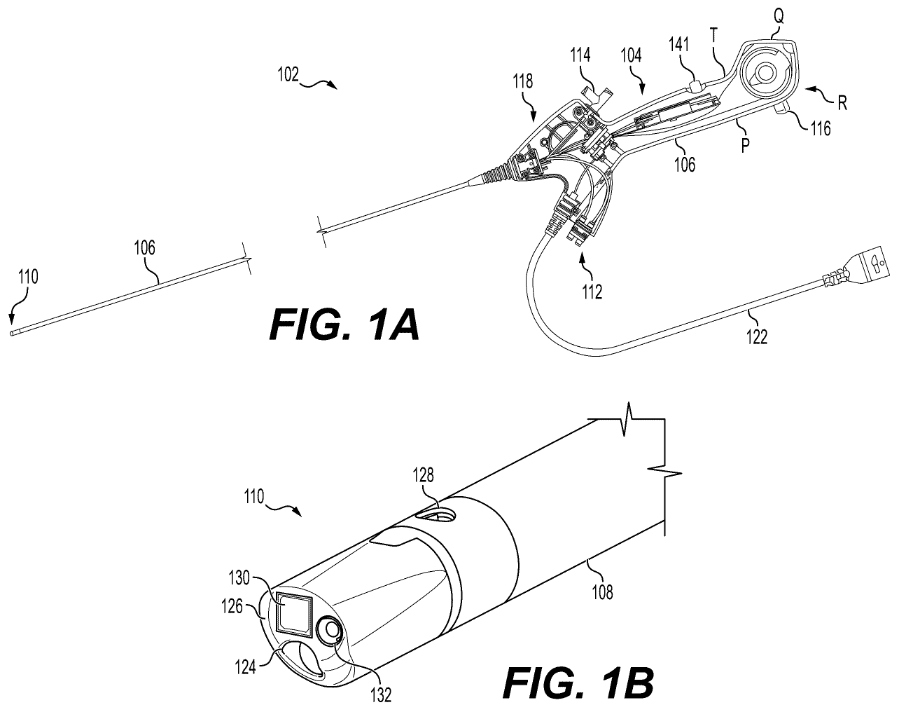

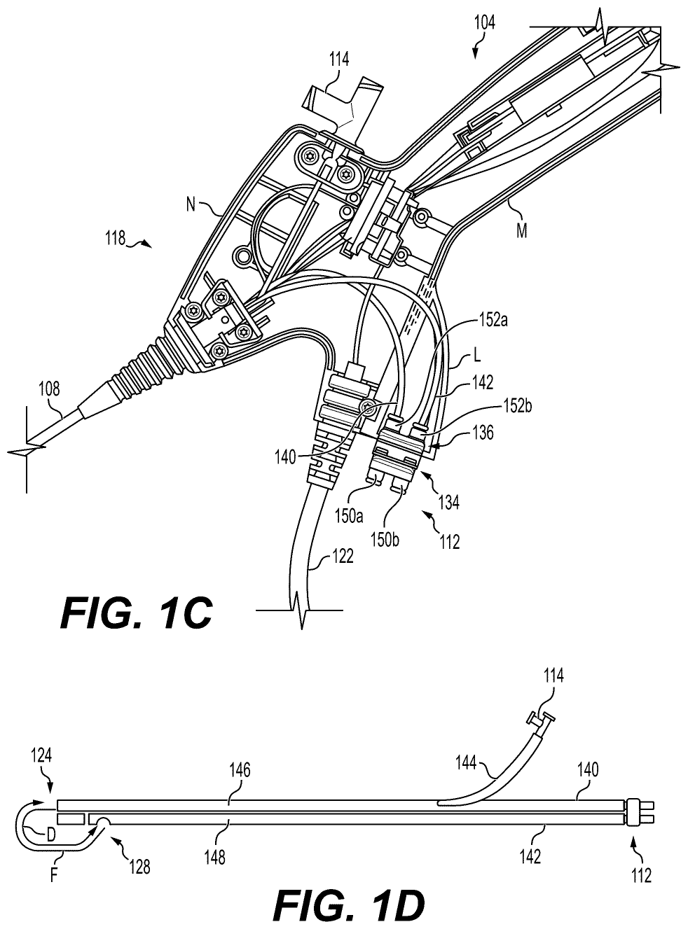

An exemplary medical device 102 is shown in FIGS. 1A-1D. Medical device 102 includes a handle 104 including a housing 106, and a shaft 108 extending distally from housing 106. Shaft 108 may be at least partially flexible to facilitate navigation through tortuous anatomical passages in the body. Shaft 108 may include a distal end portion that terminates at a distal tip 110, further illustrated in FIG. 1B. Medical device 102 may include imaging components such as an imaging device 130 (e.g., camera, imager, etc.) and one or more light sources 132 (e.g., LEDs, fiber optics, etc.) at distal tip 110. Shaft 108 may include a first lumen 146 terminating at a distal opening 124 on a distalmost face 126 of distal tip 110 and a second lumen 148 terminating at an opening 128 proximal to opening 124, e.g., a side-facing opening on a radially outer side of the distal end portion of shaft 108. Opening 128 of second lumen 148 may be proximal to opening 124 of first lumen 146 and to the imaging components (e.g., imaging device 130 and one or more light sources 132). First lumen 146 may have substantially a same cross-sectional area as second lumen 148. Alternatively, first lumen 146 may have a greater or smaller cross-sectional area than second lumen 148.

Handle 104 may include a port 114 in communication with first lumen 146 of shaft 108, e.g., for insertion of a medical instrument and/or supply of fluid. In some aspects, port 114 may be a T-shaped port. Handle 104 may be coupled to one or more electronic auxiliary devices, such as, e.g., a controller, a processor, an imaging system, a display, a power supply, etc., via an umbilicus 122.

Handle 104 may include one or more actuators for controlling aspects of medical device 102, e.g., via user input. The one or more actuators may include, for example, a lever, switch, button, knob, and/or any other suitable type of mechanism for receiving and transmitting user input. For example, housing 106 may include an actuator 116 to facilitate articulation, steering, deflection, and/or other movement of shaft 108 and distal tip 110, e.g., via one or more articulation wires or other control elements extending through shaft 108. Additionally or alternatively, housing 106 may include one or more actuators 141 configured to control imaging components of the distal tip 110, e.g., to control light sources 132 and/or capture video and/or still images using imaging device 130 at distal tip 110.

Housing 106 is coupled to a switch assembly 112, which is in turn coupled to first lumen 146 and second lumen 148 of shaft 108. Switch assembly 112 is configured to facilitate, e.g., delivery of fluid(s) and/or suction to a target site within a patient's body.

FIGS. 1A and 1C show switch assembly 112 coupled to housing 106 at or proximate a distal end 118 of handle 104, and adjacent to umbilicus 122. However, it will be appreciated that switch assembly 112 may be coupled to other portions of housing 106. For example, while FIG. 1C shows switch assembly 112 coupled to housing 106 at a location marked L, the switch assembly 112 alternatively could be coupled at location M or N in FIG. 1C or at a location marked P, Q, R, or T shown in FIG. 1A. Switch assembly 112 may include a first body 134 and a second body 136 movable relative to each other. First body 134 may include or be coupled to a first inlet 150a and a second inlet 150b. First inlet 150a and second inlet 150b may each be configured to couple to a source of fluid or a source of suction. Second body 136 may include or be coupled to a first outlet 152a and a second outlet 152b, wherein first outlet 152a is coupled to first lumen 146 and second outlet 152b is coupled to second lumen 148. First outlet 152a may be in communication with first lumen 146 and second outlet 152b may be in communication with second lumen 148, e.g., via respective first tube 140 and second tube 142, each defining a channel within housing 106. For example, first tube 140 may receive or be received by first outlet 152a and second tube 142 may be receive or be received by second outlet 152b. First body 134 may be rotatable relative to second body 136. For example, an operator may rotate first body 134 relative to second body 136 to change the position of the inlets relative to the outlets.

FIG. 1D illustrates a schematic view of the flow paths provided by first and second lumens 146, 148. As shown and described above, first lumen 146 may be in communication with port 114, providing an ability to use first lumen 146 for delivery of an instrument (or fluid) via port 114 of handle 104, in addition to delivery of fluid or suction via switch assembly 112. The channel of first tube 140 may merge or otherwise be in communication with first lumen 146 and the channel of second tube 142 may merge or otherwise be in communication with second lumen 148. Port 114 may be in communication with first lumen 146, e.g., via a channel 144 within housing 106 (which optionally may be defined by a third tube or via walls of port 114 that define channel 144). Channel 144 may merge or otherwise be in communication with first lumen 146.

Switch assembly 112 is shown in a first configuration in FIGS. 1A, 1C, and 1D, in which first inlet 150a is in communication with first outlet 152a and first lumen 146, and in which second inlet 150b is in communication with second outlet 152b and second lumen 148. First body 134 may be configured to rotate 180 degrees relative to second body 136 to a second configuration of switch assembly 112, in which first inlet 150a is in communication with second outlet 152b and second lumen 148, and in which second inlet 150b is in communication with first outlet 152a and first lumen 146.

During an exemplary medical procedure, first inlet 150a may be coupled to a source of fluid and second inlet 150b may be coupled to a source of suction. In the first configuration of switch assembly 112, a fluid may be delivered from the source of fluid to a target site through first lumen 146 and suction may be applied from the source of suction to the target site through second lumen 148. An operator may rotate first body 134 relative to second body 136 (e.g., a rotation of 180 degrees) to change switch assembly 112 to the second configuration. In the second configuration, the fluid and/or the one or more medical instruments may be delivered to a target site through second lumen 148 and suction may be applied to the target site through first lumen 146. In the first configuration, fluid may flow out of opening 124 of first lumen 146 and into opening 128 of second lumen 148, e.g., in a direction D. In the second configuration, fluid may flow out of opening 128 of second lumen 148 and into opening 124 of first lumen 146, e.g., in a direction E.

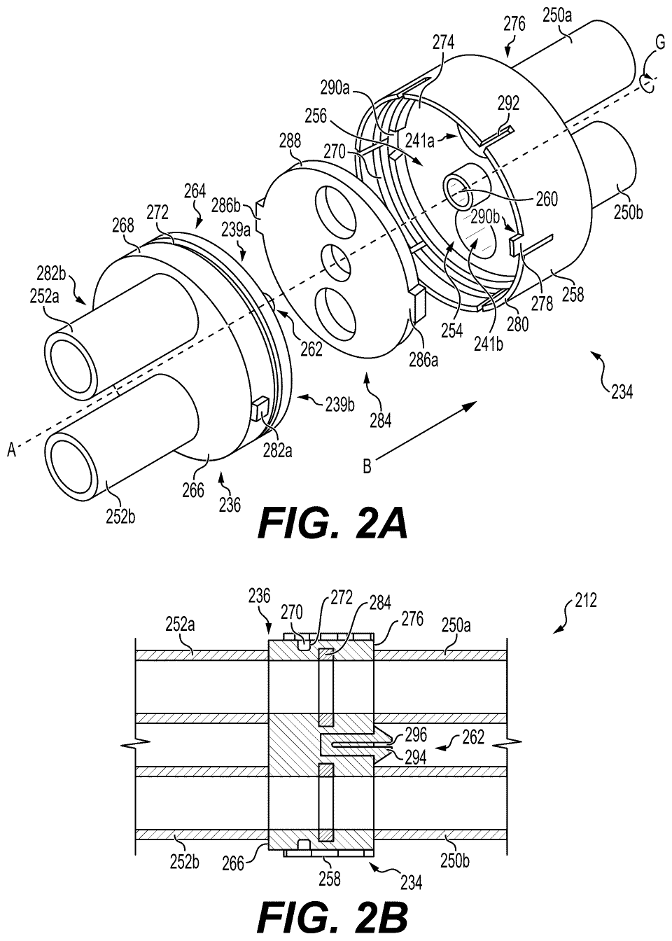

FIGS. 2A and 2B illustrate features of an exemplary switch assembly 212 that may include any of the features of switch assembly 112 discussed above. Further, switch assembly 212 may be incorporated into FIGS. 1A-1D in place of switch assembly 112. FIG. 2A illustrates an exploded view of switch assembly 212. Switch assembly 212 may include a first body 234 with a first inlet 250a and a second inlet 250b, and a second body 236 with a first outlet 252a and a second outlet 252b.

First body 234 may include a cylindrical recess 254 configured to receive portions of second body 236. Second body 236 may have a cylindrical profile to allow first body 234 to rotate relative to second body 236. For example, an outer diameter of second body 236 may be less than an outer diameter of first body 234. Second body 236 may include a first side 264 facing recess 254, a second side 266 facing away from recess 254, and a radially outer side 268. Second body 236 may include lumens 239a, 239b extending from first side 264 to second side 266 in communication with first and second outlets 252a, 252b, respectively. Recess 254 of first body 234 may include a first wall 256 and a second wall 258 surrounding recess 254. First body 234 may include lumens 241a, 241b extending from a first side 274 of first wall 256 to a second side 276 of first wall 256 in communication with first and second inlets 250a, 250b, respectively.

First body 234 may further include an aperture 260 configured to receive a corresponding protrusion 262 of second body 236 to facilitate securing second body 236 to first body 234. Protrusion 262 may extend perpendicularly from first side 264 of second body 236. In this example, a central longitudinal axis A of switch assembly 212 extends through aperture 260 and protrusion 262, and may also be the axis first body 234 rotates about. First inlet 250a, second inlet 250b, and aperture 260 of first body 234 may be arranged such that a central longitudinal axis of each of first inlet 250a, second inlet 250b, and aperture 260 are parallel to one another. Similarly, first outlet 252a, second outlet 252b, and protrusion 262 of second body 236 may be arranged such that a central longitudinal axis of each of first outlet 252a, second outlet 252b, and protrusion 262 are parallel to one another. First body 234 may further include a protrusion 270 (which may have an annular shape) extending from second wall 258 and into recess 254. Second body 236 may include a groove 272 (which may have a shape complementary to protrusion 270, e.g., an annular shape) formed on radially outer side 268 and configured to receive or otherwise engage protrusion 270 of first body 234. Engagement between protrusion 270 and groove 272 may allow for rotational movement of first body 234 relative to second body 236 while preventing axial movement of first body 234 relative to second body 236.

First body 234 and second body 236 may include stop features to limit rotation relative to each other, e.g., to facilitate aligning inlets of first body 234 with outlets of second body 236. In some examples, first body 234 may include a tab 278 extending from an outer edge 280 of second wall 258, and configured to contact first stop 282a or second stop 282b of second body 236 as first body 234 rotates. First and second stops 282a, 282b may extend radially outward from radially outer side 268 of second body 236. In some examples, first body 234 may be rotatable relative to second body 236 by at least 90 degrees, at least 120 degrees, or at least 180 degrees, e.g., an angle ranging from about 90 degrees to 180 degrees. In at least one example, second stop 282b may be positioned approximately 180-200 degrees, e.g., about 190 degrees, relative to first stop 282a allowing first body 234 to rotate about 180-200 degrees relative to second body 236.

Switch assembly 212 may include a gasket 284 to form a fluid-tight seal between first body 234 and second body 236. Gasket 284 may be received within recess 254, and may include locking features to secure gasket 284 to first body 234. For example, locking features may include first and second projections 286a, 286b extending radially outward from opposite sides on a radially outer side 288 of gasket 284. Gasket 284 may include openings that align with aperture 260, lumen 241a, and lumen 241b of first body 234. First and second projections 286a, 286b may be configured to fit within respective slots 290a, 290b within recess 254 to prevent gasket 284 from rotating relative to first body 234, allowing gasket 284 to rotate with first body 234 relative to second body 236. Alternatively or additionally, gasket 284 may be fixed to recess 254, e.g., via an adhesive or friction fit.

FIG. 2B illustrates a schematic cross-sectional view of switch assembly 212 in a first configuration, in which first inlet 250a is in communication with first outlet 252a, and in which second inlet 250b is in communication with second outlet 252b. During an exemplary assembly of switch assembly 212, gasket 284 may be secured within recess 254 by positioning first and second projections 286a, 286b within respective slots 290a, 290b of first body 234. Second body 236 may then be secured to first body 234 by inserting and/or extending protrusion 262 into aperture 260 (e.g., in a direction B) and inserting protrusion 270 into groove 272. Consequently, first side 264 of second body 236 may compress gasket 284 against first wall 256 of first body 234 to form a seal. Second wall 258 may include a plurality of relief slots 292 (shown in FIG. 2A) to allow second wall 258 to flex radially outward when inserting protrusion 270 into groove 272. Protrusion 262 of second body 236 may include a tip 294 having a cross-sectional area greater than remaining portions of protrusion 262 to further facilitate locking or securing second body 236 to first body 234. For example, protrusion 262 may include a relief slot 296 to allow tip 294 of protrusion 262 to flex radially inward when inserted into aperture 260 and to flex radially outward when extended beyond second side 276 of first wall 256, such that tip 294 may be prevented from moving back (e.g., in a direction opposite of direction B) into aperture 260.

In the first configuration, tab 278 may contact and/or rest against first stop 282a. To transition switch assembly 212 to a second configuration in which first inlet 250a is in communication with second outlet 252b, and in which second inlet 250b is in communication with first outlet 252a, first body 234 may be rotated in a direction G relative to second body 236 until tab 278 contacts and/or rests against second stop 282b. To transition switch assembly 212 back to the first configuration, first body 234 may be rotated in a direction opposite of direction G relative to second body 236 until tab 278 contacts and/or rests against first stop 282a. In the first configuration, first inlet 250a and first outlet 252a may be in communication with first lumen 146, and second inlet 250b and second outlet 252b may be in communication with second lumen 148 (see FIG. 1D). In the second configuration following rotation of first body 234 and second body 236 relative to each other, first inlet 250a and second outlet 252b may be in communication with second lumen 148, and second inlet 250b and first outlet 252a may be in communication with first lumen 146. In this way, the user may control which of first lumen 146 and second lumen 148 delivers fluid and which delivers suction, without disrupting a connection of fluid source and suction source to inlets 250a, 250b. It will be appreciated that other configurations are contemplated herein wherein inlets and outlets are not aligned, thus blocking fluid communication between inlets and outlets, depending on rotational position between first and second bodies 234, 236.

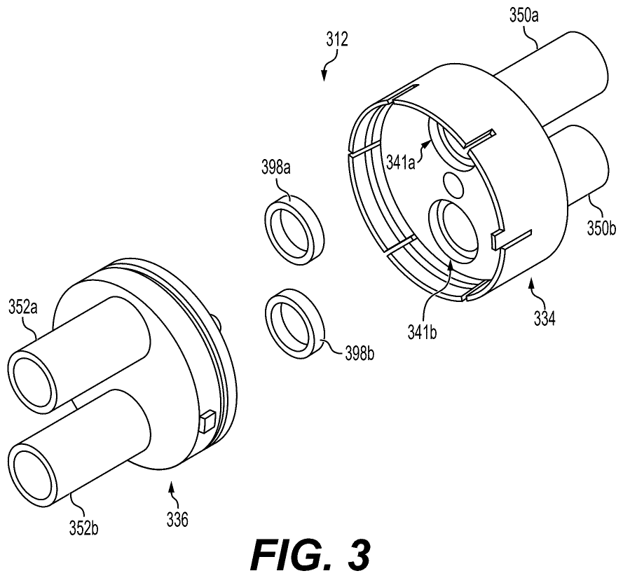

FIG. 3 illustrates an exploded view of another exemplary switch assembly 312 according to the present disclosure. Switch assembly 312 is similar to switch assembly 212 of FIGS. 2A and 2B, replacing the gasket with a sealing element, e.g., O-rings, to provide a fluid-tight seal. In this example, switch assembly 312 includes a first O-ring 398a and a second O-ring 398b to form a fluid-tight seal between a first body 334 with a first inlet 350a and a second inlet 350b, and a second body 336 with a first outlet 352a and a second outlet 352b. As shown in FIG. 3, O-rings 398a, 398b are within respective lumens 341a, 341b adjacent first and second inlets 350a, 350b.

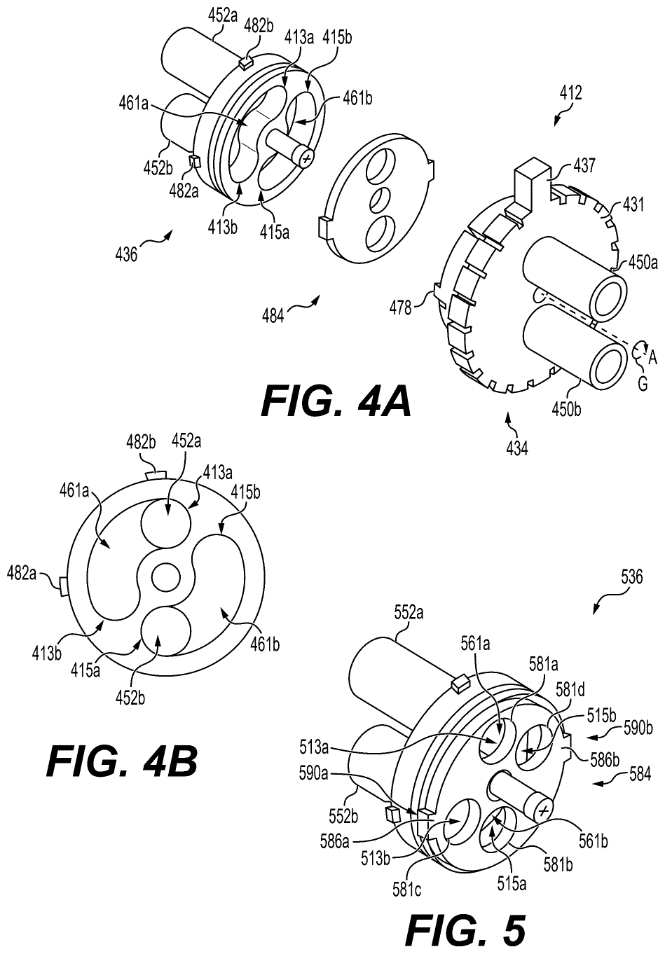

FIGS. 4A and 4B illustrate features of another exemplary switch assembly 412 that may include any of the features of switch assemblies 112, 212, 312. Switch assembly 412 may include a first body 434 with a first inlet 450a and a second inlet 450b, a second body 436 with a first outlet 452a and a second outlet 452b, and a gasket 484. FIG. 4A illustrates an exploded view of switch assembly 412 in a first configuration, in which first inlet 450a is in communication with first outlet 452a (which can be in communication with first lumen 146; see FIG. 1D), and in which second inlet 450b is in communication with second outlet 452b (which can be in communication with second lumen 148; see FIG. 1D). In this example, first body 434 may be configured to rotate less than 180 degrees, e.g., about 60 degrees to about 90 degrees, such as about 70 degrees, relative to second body 436 to transition switch assembly 412 from the first configuration to a second configuration in which first inlet 450a is in communication with second outlet 452b (which can be in communication with second lumen 148), and in which second inlet 450b is in communication with first outlet 452a (which can be in communication with first lumen 146). Optionally, first body 434 may include a wheel 431 and/or a lever 437 to facilitate rotating first body 434 relative to second body 436. For example, wheel 431 and/or lever 437 may extend from outside a slot formed on housing 106 of handle 104, which may allow an operator to rotate first body 434 relative to second body 436 by contacting wheel 431 and/or lever 437.

Second body 436 may include a first recess 461a in communication with first outlet 452a and a second recess 461b in communication with second outlet 452b. First and second recesses 461a, 461b each may have a curved or arched shape. First recess 461a may include a first end 413a and a second 413b, and first end 413a may include an opening to or of first outlet 452a. Second recess 461b may include a first end 415a and a second end 415b, and first end 415b may include an opening to or of second outlet 452b. Ends 413a, 413b of first recess 461a may be positioned adjacent to ends 415b, 415a of second recess 461b, respectively, such that less rotation of first body 434 relative to second body 436 may transition switch assembly 412 between the first and second configurations. For example, second body 436 may include a first stop 482a and a second stop 482b positioned a fixed angle (e.g., about 70 degrees to about 90 degrees, such as about 80 degrees) from first stop 482a. Additionally, first body 434 may include a tab 478 configured to rotate between first and second stops 482a, 482b.

FIG. 4B illustrates a front-facing view of second body 436. In the first configuration, tab 478 may contact and/or rest against first stop 482a, such that first inlet 450a is aligned with first outlet 452b, and second inlet 450b is aligned with second outlet 452b. To transition switch assembly 412 to the second configuration, first body 434 may be rotated in direction G relative to second body 436 until tab 478 contacts and/or rests against second stop 482b. In the second configuration, first inlet 450a may be aligned with second end 415b of second recess 461a, such that first inlet 450a is in communication with second outlet 452b and second inlet 450b may be aligned with second end 413b of first recess 461a, such that second inlet 450b is in communication with first outlet 452a.

FIG. 5 illustrates features of an exemplary second body 536, which may be used in combination with any of the features of switch assembly 412 discussed above. Second body 536 is similar to second body 436 of FIGS. 4A and 4B, but includes a recess and slots to receive a gasket 584. Gasket 584 may be similar to gaskets 284, 484 but includes two additional openings, and may be received within the recess of second body 536 instead of a recess of a first body, e.g., a first body similar to first body 434. Additionally, first and second projections 586a, 586b of gasket 584 may be configured to fit within respective slots 590a, 590b of second body 536. Gasket 584 may include openings 581a, 581b, 581c, and 581d. Openings 581a, 581b, 581c, and 581d may be positioned such that opening 581a may be aligned with a first outlet 552a and/or a first end 513a of a first recess 561a, opening 581b may be aligned with a second outlet 552b and/or a first end 515a of a second recess 561b, opening 581c may be aligned with a second end 513b of first recess 561a, and opening 581d may be aligned with a second end 515b of second recess 561b.

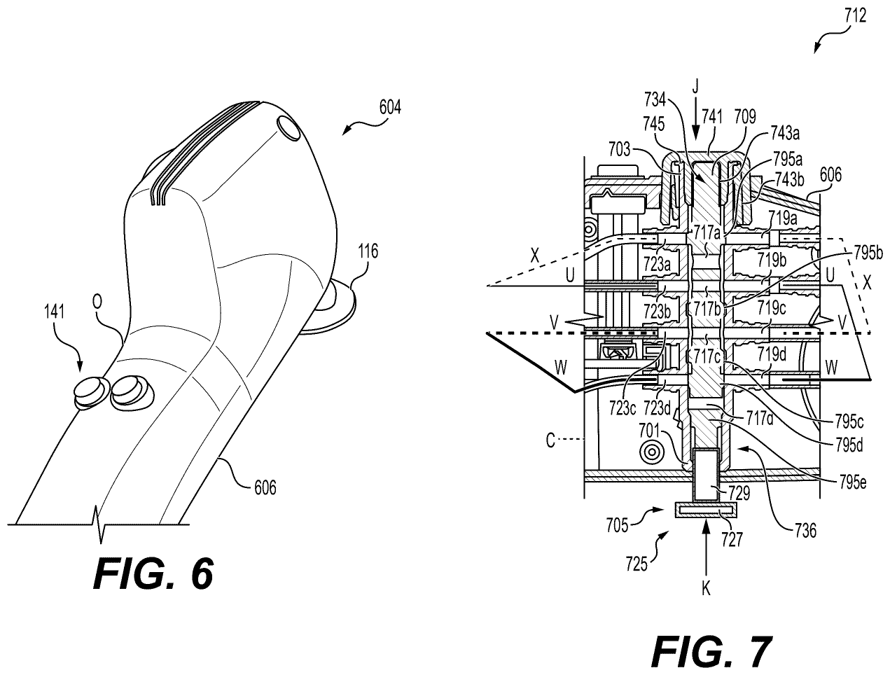

FIG. 6 illustrates features of a proximal portion of an exemplary handle 604, including a housing 606, that may be used in combination with shaft 108 of medical device 102 (e.g., in place of handle 104), or may be used in combination with any of the features of shaft 108 of medical device 102. Furthermore, handle 604 may include any of the features of handle 104 (e.g., port 114, actuators 116, 141, etc.), unless otherwise specified herein. Handle 604 may include a switch assembly coupled to first and second lumens 146, 148 of shaft 108 to facilitate, e.g., delivery of fluid(s) and/or suction to a target site within a patient's body. Exemplary switch assemblies of FIGS. 7, 8A-8C, and 9A-9C may be coupled to housing 606 at a proximal portion, e.g., a portion of housing 606 proximal to a grip configured to receive a user's hand, such as a location marked O in FIG. 6. It will be appreciated that the exemplary switch assemblies herein may be coupled to other portions of housing 606.

FIG. 7 illustrates a cross-sectional view of features of an exemplary switch assembly 712, which may include a first body 734 including a plurality of channels 717a, 717b, 717c, and 717d. According to some aspects, each channel 717a-717d may extend parallel to a longitudinal axis C of housing 606. Channels 717a-717d are configured to align with respective intermediate inlets and outlets of a second body 736. For example, second body 736 may include intermediate inlets 719a, 719b, 719c, and 719d on one side of second body 736 and may include intermediate outlets 723a, 723b, 723c, and 723d on an opposite side of second body 736. Intermediate inlets 719a, 719b, 719c, and 719d and intermediate outlets 723a, 723b, 723c, and 723d may extend parallel to longitudinal axis C. Intermediate inlets 719a, 719b, 719c, and 719d may be aligned with intermediate outlets 723a, 723b, 723c, and 723d, respectively. Additionally, first body 734 may include seals 795a, 795b, 795c, 795d, and 795e configured to block intermediate inlets 719a, 719b, 719c, 719d and intermediate outlets 723a, 723b, 723c, and 723d of second body 736 and/or isolate channels 717a-717d of first body 734 from one another. Additionally or alternatively, O-rings may be used to isolate one or more channels 717a-717d of first body 734 from one another.

In some examples, intermediate inlets 719a and 719c may be configured to couple to a single source of suction and intermediate inlets 719b and 719d may be configured to couple to a single source of fluid. For example, intermediate inlets 719a and 719c may be coupled to respective tubes within housing 606, which in turn may be coupled to a single source of fluid or a single source of suction via, e.g., a Y-connector. Similarly, intermediate inlets 719b and 719d may be coupled to respective tubes within housing 606, which in turn may be coupled to a single source of fluid or a single source of fluid via, e.g., a Y-connector. Intermediate outlets 723a and 723b may be configured to couple to a first lumen (e.g., first lumen 146 of shaft 108; see FIG. 1D) via, e.g., respective tubes within housing 606 that may merge with the first lumen. Intermediate outlets 723c and 723d may be configured to couple to a second lumen (e.g., second lumen 148 of shaft 108; see FIG. 1D) via, e.g., respective tubes within housing 606 that may merge with the second lumen.

Second body 736 may be fixed to housing 606 and may extend through and outside of housing 606. For example, second body 736 may extend from a first end 701 positioned within housing 606 to a second end 703 positioned outside of housing 606. First body 734 may be axially movable relative to second body 736, e.g., within a cavity defined by second body 736. First body 734 may include or be coupled to a cap 741 at a first end 709 of first body 734. For example, first end 709 of first body 734 may be fixed within a first recessed portion 743a of cap 741. That is, cap 741 moves axially with first body 734 relative to second body 736. Cap 741 may further include a second recessed portion 743b configured to receive second end 703 of second body 734 and including a stop surface 745. Furthermore, first body 734 may include or be coupled to a T-shaped member 725 at a second end 705 of first body 734. T-shaped member 725 may include a horizontal portion 727 (e.g., a portion extending parallel to longitudinal axis C) and a vertical portion 729 (e.g., a portion extending perpendicular to longitudinal axis C). Cap 741 and T-shaped member 725 of first body 734 may extend outside of housing 606, e.g., extend outside of respective openings of housing 606, such that an operator such as a medical professional may contact an outer surface of cap 741 to push first body 734 in a direction J relative to second body 736 and/or contact T-shaped member 725 to push first body 734 in the direction K relative to second body 736.

Switch assembly 712 is shown in a first configuration in which a source of one of fluid or suction is in communication with a first lumen (e.g., first lumen 146) via a flow path U, and in which the source of the other of fluid or suction is in communication with a second lumen (e.g., second lumen 148) via a flow path V. In the first configuration, channel 717b may be aligned with intermediate inlet 719b and intermediate outlet 723b, and channel 717c may be aligned with intermediate inlet 719c and intermediate outlet 723c. Furthermore, in the first configuration, channel 717a may be out of alignment with intermediate inlet 719a and intermediate outlet 723a, and channel 717d may be out of alignment with intermediate inlet 719d and intermediate outlet 723d. For example, seal 795a may block intermediate inlet 719a and intermediate outlet 723a, and seal 795d may block intermediate inlet 719d and intermediate outlet 723d.

To transition switch assembly 712 to a second configuration in which the source of fluid and suction are exchanged, first body 734 may be moved axially in the direction K relative to second body 736, e.g., by pushing on T-shaped member 725, until horizontal portion 727 contacts an outer surface of housing 606. Moving first body 734, including cap 741, in the direction K relative to second body 736 may move stop surface 745 of cap 741 away from second end 703 of second body 736. In the second configuration, channel 717a may be aligned with intermediate inlet 719a and intermediate outlet 723a such that the source of the other of fluid or suction is in communication with the first lumen via a flow path X, and channel 717d may be aligned with intermediate inlet 719d and intermediate outlet 723d such that the source of one of fluid or suction is in communication the second lumen via a flow path W. Furthermore, in the first configuration, channel 717b may be out of alignment with intermediate inlet 719b and intermediate outlet 723b, and channel 717c may be out of alignment with intermediate inlet 719c and intermediate outlet 723c. For example, seal 795b may block intermediate inlet 719b and intermediate outlet 723b, and seal 795c may block intermediate inlet 719c and intermediate outlet 723c.

To transition switch assembly 712 back to the first configuration, first body 734 may be moved axially in the direction J relative to second body 736, e.g., by pushing on cap 741, until stop surface 745 of cap 741 contacts second end of 703 of second body 736.

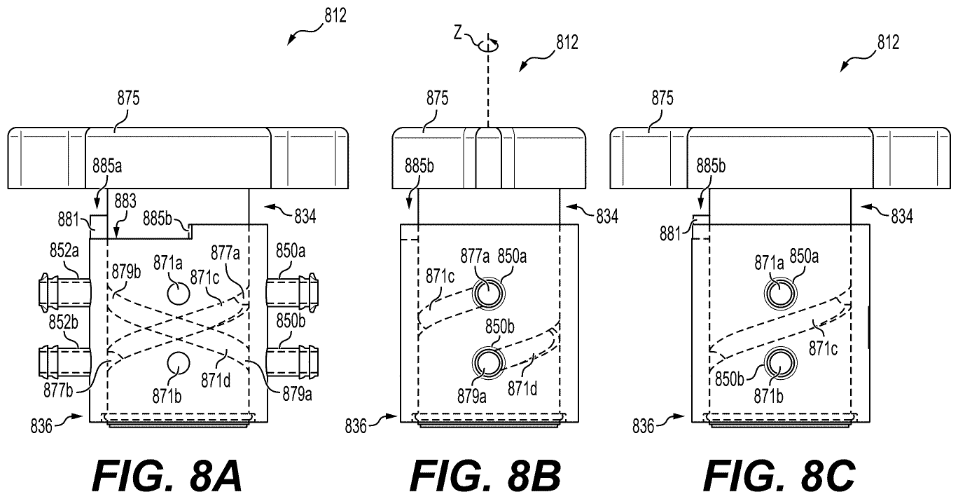

FIGS. 8A-8C illustrate features of another exemplary switch assembly 812 including a first body 834 that defines a plurality of channels configured to be in fluid communication with inlets and outlets on a second body 836 of switch assembly 812. Second body 836 may be positioned within housing 606 and first body 834 may be received within a cavity defined by second body 836. First body 834 may be coupled to or include a knob 875 positioned outside of housing 606 of handle 604, e.g., outside of an opening formed on housing 606, to allow an operator to rotate first body 834 in a direction Z relative to second body 836.

As shown in FIG. 8A, first body 834 may include first and second channels 871a, 871b extending through first body 834 and parallel to one another. First body 834 may further include third and fourth channels 871c, 871d extending along an outer surface of first body 834. For example, third and fourth channels 871c, 871d may be defined by helical grooves formed on the outer surface of first body 834. Channels 871a, 871b, 871c, and 871d may be arranged such that ends of channels 871a, 871b may be positioned approximately 90 degrees from ends of third and fourth channels 871c, 871d. Furthermore, ends of third and fourth channels 871c, 871d may be parallel to one another. For example, ends 877a, 877b of third channel 871c may be parallel to ends 879a, 879b of fourth channel 871d.

Second body 836 may include first and second inlets 850a, 850b, and may further include first and second outlets 852a, 852b. First and second inlets 850a, 850b and first and second outlets 852a, 852b may be positioned on opposite sides of second body 836. First and second inlets 850a, 850b of second body 836 may each be configured to couple to a source of suction or a source of fluid. First outlet 852a may be coupled to first lumen 146 of shaft 108 and second outlet 852b may be coupled to second lumen 148 of shaft 108.

Switch assembly 812 is shown in a first configuration in FIGS. 8A and 8B. In the first configuration, first inlet 850a may be in communication with second outlet 852b (which can be in communication with second lumen 148 via third channel 871c, and second inlet 850b may be in communication with first outlet 852a (which can be in communication with first lumen 146) via fourth channel 871d. As shown in FIG. 8B, first inlet 850a may be aligned with end 877a of third channel 871c and second inlet 850b may be aligned within end 879a of fourth channel 871d. On the opposite side of switch assembly 812, first outlet 852a may be aligned with end 879b of fourth channel 871d and second outlet 852b may be aligned with end 877b of third channel 871c. First body 834 may further include a projection 881 extending radially outward from first body 834 and positioned adjacent to knob 875. Projection 881 may be configured to move within a notch 883 of second body 836 from a first end 885a of notch 883 to a second end 885b of notch 883. In the first configuration, projection 881 may be positioned at end 885a of notch 883.

First body 834 may be rotated in a direction Z relative to second body 836, e.g., by rotating knob 875, until projection 881 contacts second end 885b of notch 883 to transition switch assembly 812 to a second configuration shown in FIG. 8C, in which first inlet 850a may be in communication with first outlet 852a (e.g., and in communication with first lumen 146) via first channel 871a, and second inlet 850b may be in communication with second outlet 852b and second lumen 148 via second channel 871b. In the second configuration, first inlet 850a and first outlet 852a may be aligned with first channel 871a, and second inlet 850b and second outlet 852b may be aligned with second channel 871b. To transition switch assembly 812 back to the first configuration, first body 834 may be rotated in a direction opposite of direction Z relative to second body 836 until projection 881 contacts first end 885a of notch 883.

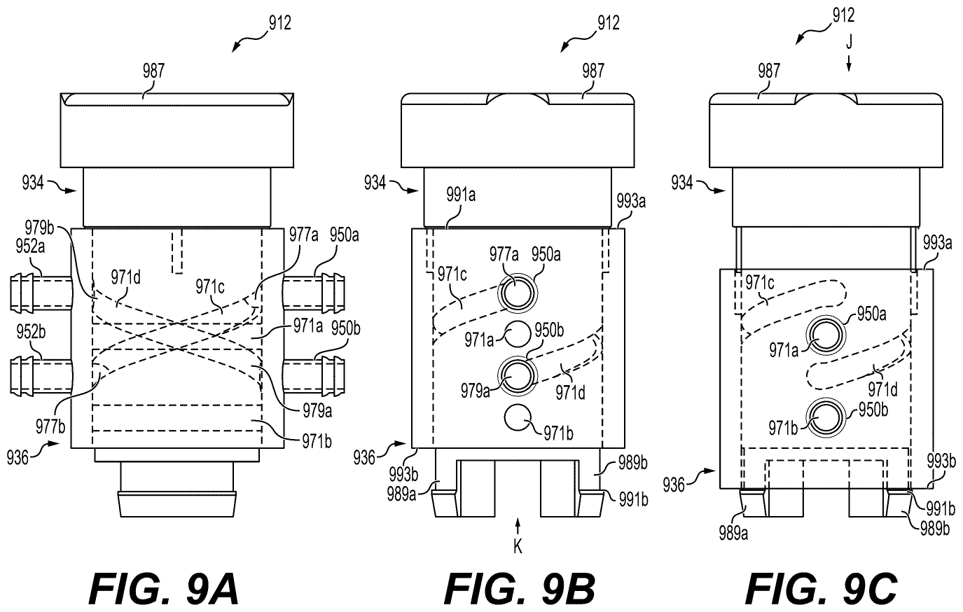

FIGS. 9A-8C illustrate features of another exemplary switch assembly 912. Similar to switch assembly 812, switch assembly 912 may include a first body 934 including first and second channels 971a, 971b extending through first body 934 and parallel to one another, and including third and fourth channels 971c, 971d extending along an outer surface of first body 934. For example, third and fourth channels 971c, 971d may be defined by helical grooves formed on the outer surface of first body 934. In this example, channels 971a, 971b, 971c, and 971d may be positioned on first body 934 such that ends of channels 971a, 971b, 971c, and 971d may be parallel to one another. Similar to switch assembly 812, switch assembly 912 may include a second body 936 including first and second inlets 950a, 950b, each configured to couple to a source of suction or a source of fluid, and may include a first outlet 952a coupled to first lumen 146 of shaft 108, and a second outlet 952b coupled to second lumen 148 of shaft 108. Similar to second body 836, second 936 may be positioned within housing 606, and first body 934 may be received within a cavity defined by second body 936.

First body 934 may be axially movable relative to second body 936. First body 934 may be coupled to or include a user interface 987, e.g., a knob or button, positioned outside of housing 606 of handle 604, e.g., outside of an opening formed on housing 606, to allow an operator to move first body 934 in direction J relative to second body 936. First body 934 may further include arms 989a, 989b at an opposite end of user interface 987. Arms 989a, 989b may be positioned outside of housing 606 of handle 604, e.g., outside of an opening formed on housing 606, to allow an operator to move first body 934 in direction K relative to second body 936. Additionally, arms 989a, 989b may be configured to flex radially inward to facilitate placing first body 934 within the cavity of second body 936.

Switch assembly 912 is shown in a first configuration in FIGS. 9A and 9B. In the first configuration, first inlet 950a may be in communication with second outlet 952b (which can be in communication with second lumen 148) via third channel 971c, and second inlet 950b may be in communication with first outlet 952a (which can be in communication with first lumen 146) via fourth channel 971d. As shown in FIG. 9B, first inlet 950a may be aligned with an end 977a of third channel 971c and second inlet 950b may be aligned with an end 979a of fourth channel 971d. On the opposite side of switch assembly 912, first outlet 952a may be aligned with an end 979b of fourth channel 971d and second outlet 952b may be aligned with an end 977b of third channel 971c. In the first configuration, a first end 993a of second body 936 may contact a first ledge 991a of first body 934.

First body 934 may be moved in direction K relative to second body 936, e.g., by pushing on arms 989a, 989b, until a second end 993b of second body 936 contacts a second ledge 991b of first body 934 to a second configuration shown in FIG. 9C in which first inlet 950a may be in communication with first outlet 952a (e.g., and first lumen 146) via first channel 971a, and second inlet 950b may be in communication with second outlet 952b (e.g., and second lumen 148) via second channel 971b. In the second configuration, first inlet 950a and first outlet 952a may be aligned with first channel 971a, and second inlet 950b and second outlet 952b may be aligned with second channel 971b. To transition switch assembly 912 back to the first configuration, first body 934 may be moved axially in direction J relative to second body 936, e.g., by pushing on user interface 987, until first end 993a of second body 936 contacts first ledge 991a of first body 934.

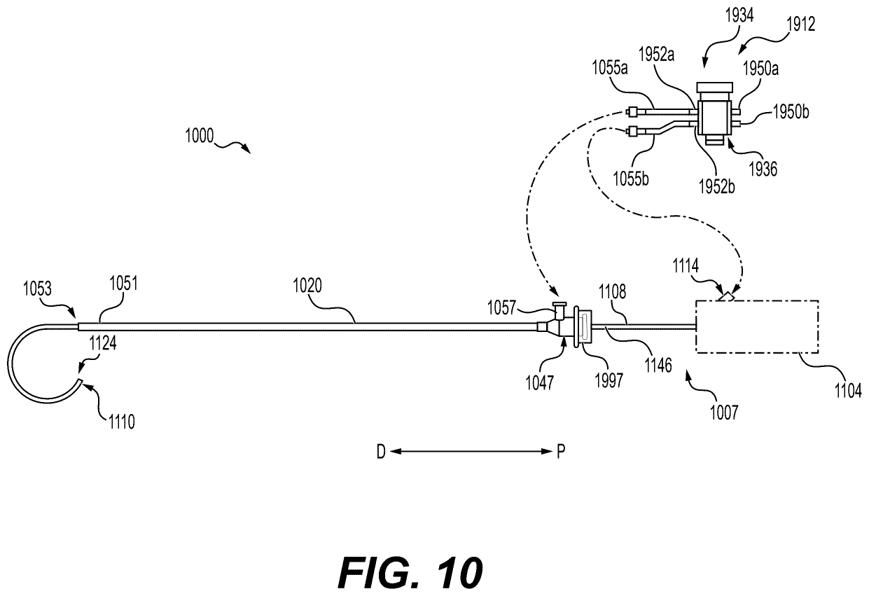

FIG. 10 illustrates a schematic view of an exemplary medical system 1000 coupled to a switch assembly 1912 according to some aspects of the present disclosure. Switch assembly 1912 may be similar to and/or include any of the features of switch assemblies 812, 912 described above, and may be used to deliver, e.g., fluid and/or suction to a target site within a patient's body. In this example, the switch assembly 1912 may be coupled to portions of a medical device other than a handle of the medical device.

Medical system 1000 may include an insertion device 1007 including a handle 1104 and a shaft 1108 extending distally from handle 1104, the shaft 1108 terminating at a distal end 1110. Handle 1104 may include any of the features of handles 104, 604, and shaft 1108 may include any of the features of shaft 108, unless otherwise specified herein. Shaft 1108 may include a first lumen 1146 terminating at distal opening 1124, which may be at distal end 1110. Handle 1104 may include a port 1114 in communication with first lumen 1146.

Medical system 1000 may include a sheath 1020 at least partially surrounding shaft 1108, such that sheath defines a second lumen 1051 concentric with first lumen 1146. Sheath 1020 may have a greater cross-sectional area than shaft 1108, such that a space is formed between an inner surface of sheath 1020 and an outer surface of shaft 1108 (thus defining second lumen 1051) when shaft 1108 is received within sheath 1020. Second lumen 1051 may extend to a distal end 1053 of sheath 1020. Sheath 1020 may include or be coupled to a connector 1047 (which may be configured as a hub) at a proximal end of sheath 1020, and connector 1047 may provide fluid communication with second lumen 1051 via a port 1057. Shaft 1108 of insertion device 1007 may extend through connector 1047 and may be movable relative to connector 1047 and sheath 1020.

Fluid or suction may be delivered through second lumen 1051 exterior of shaft 1108, e.g., supplied via port 1057. Additionally, medical system 1000 may include a seal 1997 to seal the proximal end of sheath 1020 to connector 1047 and around shaft 1108.

While FIG. 10, shows distal end 1053 of sheath 1020 proximal of distal end 1110 of shaft 1108, shaft 1108 may be movable relative to sheath 1020 as mentioned above in a proximal direction P or a distal direction D. For example, shaft 1108 may move proximally and distally relative to sheath 1020, e.g., to align distal end 1053 of sheath 1020 with distal end 1110 of shaft 1108 or to adjust relative positions of the distal ends 1110, 1053.

As mentioned above, switch assembly 1912 may be similar to switch assembly 912. In this example, switch assembly 1912 may include or be coupled to connectors (e.g., tubes) configured to connect outlets of switch assembly 1912 to insertion device 1007 and sheath 1020. Switch assembly 1912 may include a first body 1934 similar to first body 934, and may include a second body 1936 similar to second body 936.

Switch assembly 1912 may include a first outlet 1952a coupled to a first tube 1055a that is configured to couple to connector 1047 to provide fluid communication with second lumen 1051 of sheath 1020. Switch assembly 1912 also may include a second outlet 1952b coupled to a second tube 1055b configured to couple to port 1114 of handle 1104, providing fluid communication with first lumen 1146. First tube 1055a and second tube 1055b may be detachable from first and second outlets 1952a, 1952b. Switch assembly 1912 may include first and second inlets 1950a, 1950b, each configured to couple to a source of suction or a source of fluid.

To exchange whether fluid or suction is provided through first lumen 1146 or second lumen 1051, an operator (e.g., a medical professional) may move first body 1934 relative to second body 1936 as described above for switch assembly 912 to control whether first inlet 1950a is in communication with first outlet 1952a or second outlet 1952b, and whether second inlet 1950b is in communication with first outlet 1952a or second outlet 1952b.

During an exemplary medical procedure, the operator may connect first inlet 1950a to a source of fluid and connect second inlet 1950b to a source of suction. In this configuration, the source of fluid is in communication with first lumen 1146 of shaft 1108 to deliver fluid to a target site via distal opening 1124, and the source of suction is in communication with second lumen 1051 of sheath 1020 to deliver suction to the target site via an opening at distal end 1053. The operator may optionally move shaft 1108 and/or sheath 1020 so that distal end 1110 of shaft 1108 is proximal to distal end 1053 of sheath 1020, which may form a funnel distal of distal end 1110 of shaft 1108 when fluid and suction are supplied simultaneously. To transition from delivering fluid through first lumen 1146 and suction through second lumen 1051 of sheath 1020 to delivering suction through first lumen 1146 and fluid through second lumen 1051, the operator may move first body 1934 relative to second body 1936 (see direction K in FIG. 9B).

In some examples, only one outlet of switch assembly 1912 may be connected to first lumen 1146 or second lumen 1051, while the other outlet may be sealed or blocked. For example, switch assembly 1912 may be used to deliver fluid or suction to second lumen 1051 while one or more medical instruments may be delivered to the target site through first lumen 1146, e.g., via port 1114 of handle 1104.

It will be apparent to those skilled in the art that various modifications and variations may be made in the disclosed devices and methods without departing from the scope of the disclosure. Other aspects of the disclosure will be apparent to those skilled in the art from consideration of the specification and practice of the features disclosed herein. It is intended that the specification and embodiments be considered as exemplary only.

We claim:

1. A medical device comprising:
a handle comprising a housing;
a first lumen extending distal to the housing;
a second lumen extending distal to the housing; and
a switch assembly coupled to at least one of the first lumen or second lumen;
wherein the switch assembly includes a first inlet, a second inlet, a first outlet, and a second outlet, the first inlet being configured to receive a source of fluid and the second inlet being configured to receive a source of suction; and
wherein the switch assembly is movable between a first configuration in which the first inlet is in communication with the first outlet and the first lumen and a second configuration in which the second inlet is in communication with the first outlet and the first lumen.

2. The medical device of claim 1, wherein the medical device includes a shaft extending distally from the housing, the shaft defining the first lumen and the second lumen, wherein the switch assembly is coupled to the housing of the handle.

3. The medical device of claim 1, wherein the switch assembly includes a first body that includes the first inlet and the second inlet, and a second body that includes the first outlet and the second outlet, the first body being movable relative to the second body.

4. The medical device of claim 3, wherein the first body is rotatable relative to the second body to transition between the first configuration and the second configuration.

5. The medical device of claim 4, wherein the first body is rotatable about 60 degrees to about 180 degrees relative to the second body to transition between the first configuration and the second configuration.

6. The medical device of claim 4, wherein the first body includes a recess for receiving the second body, and wherein the recess includes a protrusion configured to engage a groove of the second body.

7. The medical device of claim 4, wherein the first body includes a first stop and a second stop, and the second body includes a tab configured to engage and move between the first stop and the second stop.

8. The medical device of claim 3, wherein the switch assembly includes a gasket configured to form a seal between the first body and the second body.

9. The medical device of claim 3, wherein the second body includes a protrusion configured to be received within an aperture of the first body to secure the second body to the first body.

10. The medical device of claim 3, wherein the first body is axially movable relative to the second body to transition the medical device between the first configuration and the second configuration.

11. The medical device of claim 10, wherein the second body includes the first inlet, the second inlet, the first outlet, and the second outlet, and wherein moving the first body relative to the second body controls fluid communication between the first inlet and one of the first outlet or the second outlet, and controls fluid communication between the second inlet and one of the first outlet or the second outlet.

12. The medical device of claim 11, wherein the first body includes a plurality of channels, wherein the first inlet is configured to couple to the first outlet and the second outlet via a first set of channels of the plurality of channels, and wherein the second inlet is configured to couple to the first outlet and the second outlet via second set of channels of the plurality of channels.

13. The medical device of claim 12, wherein at least one channel of the first set of channels and at least one of channel of the second set channels are blocked in the first configuration and in the second configuration.

14. The medical device of claim 12, wherein each of the first set of channels and the second set of channels includes a channel extending through the first body and a channel formed on an outer surface of the first body.

15. A medical device comprising:
a handle comprising a housing;
a shaft extending distally from the handle, the shaft defining a first lumen that terminates at a first opening and a second lumen that terminates at a second opening; and
a switch assembly coupled to the housing and including a first body movably coupled to a second body, wherein the switch assembly includes a first inlet configured to receive a source of a fluid, a second inlet configured to receive a source of suction, a first outlet in communication with the first lumen, and a second outlet in communication with the second lumen, and wherein the switch assembly is movable between a first configuration in which the first inlet is in communication with the first outlet and the first lumen and a second configuration in which the first inlet is in communication with the second outlet and the second lumen.

16. The medical device of claim 15, wherein the first opening is at a distalmost end of the shaft and the second opening is proximal to the first opening.

17. The medical device of claim 15, wherein the second body includes the first inlet, the second inlet, the first outlet, and the second outlet, and the first body is axially movable within the second body.

18. The medical device of claim 17, wherein the first body includes a plurality of channels parallel to each other, wherein moving the first body relative to the second body establishes fluid communication between the first inlet and the first outlet via one of the plurality of channels, and further moving the first body relative to the second body establishes fluid communication between the first inlet and the second outlet via another one of the plurality of channels.

19. A medical device comprising:
a handle comprising a housing;
a shaft extending distally from the handle, the shaft defining a first lumen that terminates at a first opening and a second lumen that terminates at a second opening proximal to the first opening; and
a switch assembly coupled to the housing and including a first body movably coupled to a second body, wherein the switch assembly includes a first inlet configured to receive a source of a fluid, a second inlet configured to receive a source of suction, a first outlet in communication with the first lumen, and a second outlet in communication with the second lumen, and wherein the switch assembly is movable between a first configuration and a second configuration by rotating or axially moving the first body relative to the second body, wherein in the first configuration, the first inlet is in communication with the first outlet and the first lumen, and wherein in the second configuration, the first inlet is in communication with the second outlet and the second lumen.

20. The medical device of claim 19, wherein the first body includes the first inlet and the second inlet, and the second body includes the first outlet and the second outlet, the first body being rotatable relative to the second body.
